(19) United States
(12) Patent Application Publication
(10) Pub. No.: US 2024/0034708 A1
(43) Pub. Date: Feb. 1, 2024

(54) HIGH PURITY HYDROXYCARBOXYLIC ACID COMPOSITIONS AND METHODS OF MAKING SAME

(71) Applicant: Solugen, Inc., Houston, TX (US)

(72) Inventors: Kevin Loftis, Spring, TX (US); Sean Hunt, Houston, TX (US); Peter Nguyen, Houston, TX (US); Parth Patel, Seabrook, TX (US)

(73) Assignee: Solugen, Inc., Houston, TX (US)

(21) Appl. No.: 18/266,213

(22) PCT Filed: Dec. 14, 2021
(86) PCT No.: PCT/US2021/063202

§ 371 Date: Jun. 8, 2023

Related U.S. Application Data
(60) Provisional application No. 63/125,306, filed on Dec. 14, 2020.

(51) Int. Cl. C07C 51/31; C12P 7/58

(52) U.S. Cl. CPC C07C 51/31; C12P 7/58

(57) ABSTRACT

A system for glucaric acid production includes (a) a first input selected from the group consisting of glucuronolactone, a disaccharide feedstock, a cleaved starch, a disaccharide, glucuronic acid, or a combination thereof. In addition, the system includes (b) a first catalyst system comprising a metal oxidation catalyst; (c) a first product; (d) a second input. Further, the system includes (e) a second catalyst system comprising an enzyme. Still further, the system includes (f) a second product comprising from about 50% to about 99% glucaric acid on a dry basis.

### CROSS-REFERENCE TO RELATED APPLICATIONS

This application is a 35 U.S.C. § 371 national stage application of PCT/US2021/063202 filed Dec. 14, 2021 and entitled “High Purity Hydroxycarboxylic Acid Compositions and Methods of Making Same,” which claims benefit of U.S. provisional patent application Ser. No. 63/125,306 filed Dec. 14, 2020, and entitled “High Purity Hydroxycarboxylic Acid Compositions and Methods of Making Same,” each of which is hereby incorporated herein by reference in its entirety for all purposes.

### STATEMENT REGARDING FEDERALLY SPONSORED RESEARCH OR DEVELOPMENT

Not applicable.

### TECHNICAL FIELD

The present disclosure relates generally to compositions and methods for the production of high purity hydroxycarboxylic acid compositions. More particularly, the present disclosure relates to chemoenzymatic methods for the production of high purity glucaric acid.

### SUMMARY

A system for glucaric acid production, the system comprising (a) a first input selected from the group consisting of glucuronolactone, a disaccharide feedstock, a cleaved starch, a disaccharide, glucuronic acid, or a combination thereof; (b) a first catalyst system comprising a metal oxidation catalyst; (c) a first product; (d) a second input; (e) a second catalyst system comprising an enzyme; and (f) a second product comprising from about 50% to about 99% glucaric acid on a dry basis.

A method for glucaric acid production, the method comprising (a) providing a first input selected from the group consisting of glucuronolactone, a disaccharide feedstock, a cleaved starch, a disaccharide, glucuronic acid, and a combination thereof; (b) contacting the first input with a first catalyst system comprising a noble metal oxidation catalyst; (c) producing a first product based on the contacting in step (b); (d) contacting a second input with a second catalyst system comprising an enzyme; and (e) producing a second product comprising from about 50% to about 99% glucaric acid on a dry basis based on the contacting in step (d).

### BRIEF DESCRIPTION OF THE DRAWINGS

For a detailed description of various exemplary aspects, reference will now be made to the accompanying drawings in which:

FIG. 1 is a schematic view of a reactor configuration in accordance with an aspect of the present disclosure.

FIG. 2 is a graph of the total conversion of the glucuronic/glucuronolactone feed on a mass basis for the samples from Example 1.

FIG. 3 is a graph of the dry weight percent content of glucuronic/glucuronolactone and glucaric acid in the process stream of Example 1.

FIG. 4 is a graph of the cumulative productivity of the reactor catalyst based on run time and catalyst exposure for the samples from Example 1.

FIG. 5 is a graph of the weight percent of products generated by the reaction described in Example 1.

### BACKGROUND

Hydroxycarboxylic acids are an important group of chemicals, with several members that are commercially manufactured in large volumes and have a wide range of applications. Some of the important uses of hydroxycarboxylic acids are related to environmentally friendly products and processes such as biodegradable plastics for consumer products; and nontoxic and easily degradable solvents, cleaning agents, plasticizers, and the like. The emergence of new technologies for efficient and economical manufacture of these chemicals, combined with these product opportunities, may result in hydroxycarboxylic acids becoming relatively large production volume chemicals of global commercial importance.

Glucaric acid is a hydroxycarboxylic acid, and in particular, a six-carbon diacid that has potential to serve as a platform chemical. Glucaric acid has utility in a variety of applications such as in the production of adipic acid for renewable nylon-6,6, as an intermediate in the production of 2,5-furandicarboxylic acid (FDCA), as a high-performance renewable replacement for polyethylene terephthalate (PET) in two-liter bottles, as a polymer additive to increase the mechanical properties of several different classes of industrial fibers, as a corrosion inhibitor, and as a reinforcing agent.

### DETAILED DESCRIPTION

Commercialization of glucaric acid has been hindered largely due to the lack of economically-viable production methods. Current production methods, such as the use of corn starch and nitric acid oxidation, are poorly selective, thereby resulting in a mixture of glucaric acid and a number of other reaction products that must then be separated. Other processes, such as fermentation, display improved selectivity for the production of glucaric acid but such processes are not very economical. Yet another common approach to glucaric acid production involves the oxidation of gluconic acid using a metal catalyst. The main obstacle in producing glucaric acid in this manner is the side product distribution from keto groups or from carbon cleavage. Thus, an ongoing need exists for methods of producing high purity hydroxycarboxylic acids (e.g., glucaric acid) that overcomes the challenges associated with traditional production processes. Accordingly, aspects described herein are directed to the production of high purity hydroxycarboxylic acids such as glucaric acid that offer the potential to overcome the shortfalls of conventional approaches to producing high purity hydroxycarboxylic acids.

Disclosed herein are methods for the production of a high purity hydroxycarboxylic acids (HCA). Although this disclosure refers to the production of high purity glucaric acid, it is to be understood this is exemplary and the production of other high purity hydroxycarboxylic acids (e.g., glycolic acid, lactic acid, etc.) are also contemplated and within the scope of this disclosure. In an aspect, production of the HCA is carried out using a system of reactors and associated equipment. Such production systems and methods disclosed herein may also be referred to herein as “high purity glucaric acid production configurations” or HIGAP.

In an aspect, a HIGAP is characterized as having a first input to a first catalyst system to produce a first product and a second input to a second catalyst system to produce a second product. In some aspects, the first input, the second input, the first product, the second product, or a combination thereof comprises a mixture of molecules or compounds. In one or more aspects, the first product and the second input are the same (e.g., the first product serves as the second input). In an alternative aspect, the first product and the second input are different. In an aspect, the first input is selected from the group consisting of glucuronolactone, a disaccharide feedstock, a cleaved starch, a disaccharide, glucuronic acid, or a combination thereof; the first catalyst system comprises a metal oxidation catalyst; the first product comprises an oxidized form of the first input; the second input comprises an oxidized form of the first input; the second catalyst comprises an enzyme; and the second product comprises glucaric acid, alternatively high purity glucaric acid.

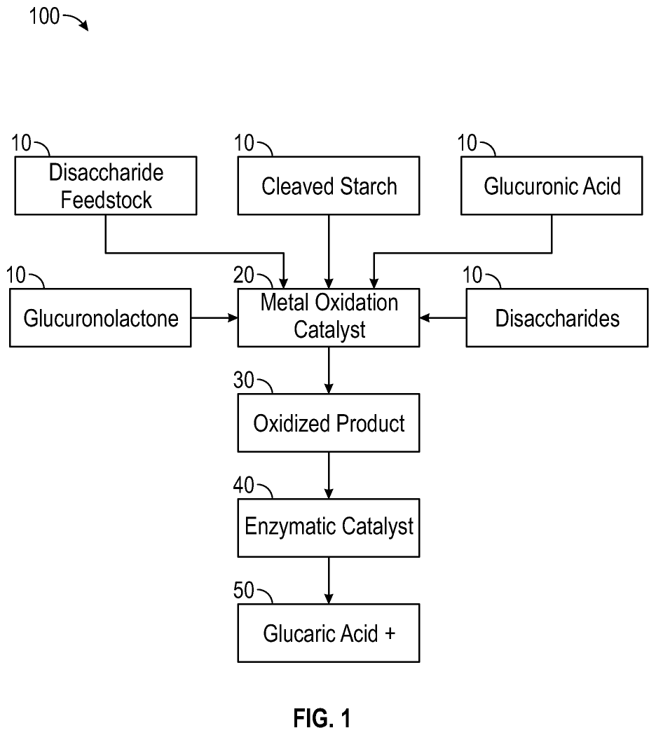

A schematic depiction of an aspect of a HIGAP 100 and associated method of the present disclosure is shown in FIG. 1. Referring to FIG. 1, the method of the present disclosure comprises introduction of a first input 10 to a first catalyst system 20. The first input 10 may be selected from the group consisting of glucuronic acid, glucuronolactone, a disaccharide feedstock, a cleaved starch, a disaccharide, or a combination thereof, while the first catalyst comprises a metal oxidation catalyst. Introduction of the first input 10 to the first catalyst system 20 can be carried out under any conditions compatible with the first input 10 and first catalyst system 20 so as to allow reaction between the first input 10 and the first catalyst system 20. Reaction of the first input 10 with the first catalyst system 20 results in a first product 30. In one or more aspects, the first product 30 is an oxidized form of the first input 10. As depicted in FIG. 1, the oxidized form of the first input 10 is introduced as a second input 30 to the second catalyst system 40. As shown in FIG. 1, the second catalyst system 40 comprises an enzymatic catalyst. Introduction of the second input 30 to the second catalyst system 40 can be carried out under any conditions compatible with the second input 30 and second catalyst system 40 so as to allow reaction between the second input 30 and second catalyst system 40 resulting in a second product (e.g., glucaric acid). Each component of the HIGAP 100 will now be described in more detail.

In an aspect, the first input 10 comprises a compound selected from the group consisting of a mixture of glucuronic acid and glucuronolactone, a disaccharide feedstock, a cleaved starch, a disaccharide, glucuronic acid, a chelant, or a combination thereof.

In an aspect, the first input 10 is glucuronic acid, glucuronolactone, or a combination thereof. The intra-ester linkage (i.e., the lactone of glucuronolactone) may spontaneously hydrolyze to the free-acid form and an equilibrium is established between glucuronolactone and glucuronic acid as depicted in Reaction I.

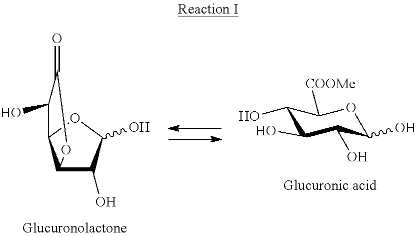

A mixture of glucuronic acid and glucuronolactone suitable for use as the first input to the HIGAP 100 may be obtained from any suitable source and/or prepared using any suitable methodology.

In an aspect, the first input 10 comprises a disaccharide feedstock. Herein, a disaccharide refers to the sugar formed when two monosaccharides are joined by a glycosidic linkage. In one or more aspects, the disaccharide feedstock comprises sucrose, lactose, maltose, isomaltose, isomaltulose, trehalose, trehalulose, or a combination thereof. In an aspect, the disaccharide feedstock comprises sucrose, lactose, maltose, or a combination thereof. In an alternative aspect, the disaccharide feedstock comprises greater than about 20 weight percent (wt. %) disaccharide, alternatively greater than about wt. % disaccharide, or alternatively from about 20 wt. % to about 50 wt. % disaccharide based on the total weight of the disaccharide feedstock, both on a dry basis. It is contemplated any composition that serves as an input (e.g., feedstock) may comprise other compounds that are compatible with the compounds and processes disclosed herein.

In an aspect, the first input 10 comprises a cleaved starch. Starch or amylum is a polymeric carbohydrate consisting of numerous glucose units joined by glycosidic bonds. A cleaved starch suitable for use as the first input 10 of the HIGAP 100 may be prepared using any suitable methodology. For example, the first input 10 may be a cleaved starch prepared by contacting a starch with a cleaving enzyme under conditions suitable for the formation of a cleaved product. Nonlimiting examples of cleaving enzymes suitable for use in the present disclosure include α-amylase, glucoamylase, β-glucuronidase, β-glucosidase, and invertase.

In an aspect, the first input 10 comprises a disaccharide. A disaccharide suitable for use in the present disclosure may be selected from the group consisting of sucrose, lactose, maltose, isomaltose, isomaltulose, trehalose, trehalulose, and combinations thereof. In such aspects, the disaccharide comprises greater than about 75%, alternatively greater than about 85% alternatively greater than about 95% of a single disaccharide, or alternatively from about 75% to about 85%, all on a dry basis. For example, the first input may comprise a material having greater than about 85% sucrose or a material having greater than about 90% maltose, both on a dry basis.

In an aspect, the first input 10 comprises a glucuronic acid composition. In such aspects, the glucuronic acid may be present in a composition in an amount of greater than about 50 wt. %, alternatively greater than about 75 wt. %, alternatively greater than about 90 wt. %, or alternatively from about 50 wt. % to about 90 wt. % based on the total weight of the composition on a dry basis.

In an aspect, the first input 10 comprises a chelant. Herein, a chelant refers to any molecule that forms two or more separate coordinate bonds between a polydentate (multiple bonded) ligand and a single central atom (e.g., metal). In an aspect, the chelant is a naturally-occurring molecule or derived from a naturally-occurring molecule such as a monosaccharide or a polysaccharide.

In an aspect, the chelant comprises aldonic acid, uronic acid, aldaric acid, or a combination thereof; and a counter cation. The counter cation may comprise an alkali metal (Group I), an alkali earth metal (Group II), or a combination thereof. In certain aspects, the counter cation is sodium, potassium, magnesium, calcium, strontium, cesium, or a combination thereof. In the alternative, the counter cation comprises aluminum, silica, titanium, or boron. In an aspect, the chelant comprises a glucose oxidation product, a gluconic acid oxidation product, a gluconate, or a combination thereof. Alternatively, the chelant comprises a buffered glucose oxidation product, a buffered gluconic acid oxidation product, or a combination thereof. In some aspects, the chelant further comprises minor component species comprising n-keto-acids, C₂-C₆ diacids, or a combination thereof.

In an aspect, the first catalyst system 20 of HIGAP 100 comprises a metal catalyst, a transition metal catalyst, a noble metal catalyst, a metal oxidation catalyst, or a combination thereof. In an aspect, the metal catalyst is a metal oxidation catalyst. In other aspects, the metal oxidation catalyst is a supported metal catalyst. In such aspects, the support comprises carbon, silica, alumina, titania (TiO₂), zirconia (ZrO₂), a zeolite, or a combination thereof. The support may contain less than about 1.0 weight percent (wt. %), alternatively less than about 0.1 wt. %, or alternatively less than about 0.01 wt. % SiO₂ binders based on the total weight of the support.

Support materials suitable for use in the present disclosure are predominantly mesoporous or macroporous, and substantially free from micropores. For example, the support may comprise less than about 20% micropores. In an aspect, the support is a porous nanoparticle support. As used herein, the term “micropore” refers to pores with diameter <2 nm, as measured by nitrogen adsorption and mercury porosimetry methods and as defined by IUPAC. As used herein, the term “mesopore” refers to pores with diameter from ca. 2 nm to ca. 50 nm, as measured by nitrogen adsorption and mercury porosimetry methods and as defined by IUPAC. As used herein, the term “macropore” refers to pores with diameters larger than 50 nm, as measured by nitrogen adsorption and mercury porosimetry methods and as defined by IUPAC.

In an aspect, the support comprises a mesoporous carbon extrudate having a mean pore diameter ranging from about 10 nm to about 100 nm and a surface area greater than about 20 m² g⁻¹ but less than about 300 m² g⁻¹. Supports suitable for use in the present disclosure may have any suitable shape. For example, the support may be shaped into 0.8-3 mm trilobes, quadralobes, or pellet extrudates. Such shaped supports enable the used of fixed trickle bed reactors to perform the final oxidation step under continuous flow.

In one or more aspects, the metal comprises one or more noble metals, alternatively a Group 8 metal (e.g., Re, Os, Ir, Pt, Ru, Rh, Pd, Ag), a 3d transition metal, an early transition metal, or a combination thereof. In an aspect, the metal oxidation catalyst comprises gold, Au.

Metal oxidation catalysts of the present disclosure may effectively oxidize the presently disclosed inputs to produce oxidation products, which may be further processed to produce compounds such as glucaric acid and derivatives thereof. In an aspect, the metal oxidation catalysts comprising platinum and gold are heterogeneous, solid-phase catalysts. In such aspects, suitable catalyst supports include, without limitation, carbon, surface treated aluminas (such as passivated aluminas or coated aluminas), silicas, titanias, zirconias, zeolites, montmorillonites, and modifications, mixtures, or a combination thereof. The catalyst support may be treated so as to promote the preferential deposition of platinum and gold on the outer surface of the support so as to create a shell type catalyst. The platinum and gold-containing catalysts that function as a metal oxidation catalyst may be produced by any suitable methodology. For example, the platinum and gold-containing catalysts may be produced using deposition procedures such as incipient wetness, ion-exchange and deposition-precipitation. In an aspect, the first product 30 is an oxidation product that is used as a second input to the second catalyst system 40.

In an aspect, the second catalyst system 40 of the HIGAP 100 comprises an enzyme. In general, any enzyme capable of catalyzing the conversion of the first product to produce a composition comprising glucaric acid may be employed. Examples of enzymes suitable for use in the second catalyst system 40 include, without limitation, (i) an oxidoreductase such as glucose oxidase (EC 1.1.3.4), catalase (EC 1.11), and combinations thereof, (ii) a hydrolase such as α-amylase (EC 3.2.1.1), glucoamylase (EC 3.2.1.2), β-glucuronidase (EC 3.2.1.31), β-glucosidase invertase (EC 3.2.1.21), and combinations thereof, (iii) an isomerase such as xylose isomerase (EC 5.3.1.5), and (iv) combinations thereof.

In an aspect, the first product 30 (i.e., the oxidized form of the first input) once contacted with the second catalyst system 40 under suitable conditions generates a second product 50 comprising glucaric acid. Glucaric acid may be present in the second product in an amount of equal to or greater than about 50%, alternatively equal to or greater than about 75%, alternatively equal to or greater than about 90%, alternatively equal to or greater than about 95%, or alternatively from about 50% to about 99%, all on a dry basis.

In an aspect, the HIGAP system disclosed herein oxidizes a disaccharide to either a carboxylic disaccharide or a dicarboxylic disaccharide utilizing a platinum and gold-containing catalyst to produce a first product that is subsequently hydrolyzed using enzymes or through metal oxidation decomposition. In the case of carboxylic disaccharide formation, this would yield one mole of glucuronolactone and one mole fructose, glucose and or gluconic acid. Using an enzyme cascade which converts the other products to gluconic acid would ideally give one mole of glucuronolactone and one mole of gluconic acid. In another aspect, a disaccharide is partially oxidized using the first catalyst system, the second catalyst system or both the first and second catalyst systems. In such aspects, partial oxidation the disaccharide results in a mixture of glucaric acid and gluconic acid.

In an aspect, enzymes utilized in the second catalyst system of the HIGAP may or may not be characterized by a high absolute specificity to the input (e.g., glucose)

In another aspect, the metal oxidation catalyst of the HIGAP may have the rate of reaction accelerated by the addition of a basic species (e.g., sodium hydroxide).

In one aspect, the first input is fructose and the first catalyst comprises xylose isomerase. In such aspects, the final product comprises glucose. In another aspect, the first input is glucose, the first catalyst is a combination of glucose oxidase and catalase and the final product comprises glucaric acid. In another aspect, the first input is a dicarboxylic disaccharide and the final product comprises at least one mole of glucuronolactone or a combination of 2-keto gluconic and 5-keto mannonic or gluconic acid. In such aspects, the final product comprises about 75% glucaric acid on a dry basis.

In an aspect, reaction products of the HIGAP system differ from those observed when using conventional methods for the production of glucaric acid. For example, the reactions disclosed herein may reduce the amount of 2-keto gluconic acid produced while increasing the amount of CO2 produced.

### EXAMPLES

The aspects having been generally described, the following examples are given as particular aspects of the disclosure and to demonstrate the practice and advantages thereof. It is understood that the examples are given by way of illustration and are not intended to limit the specification or the claims in any manner.

### Example 1

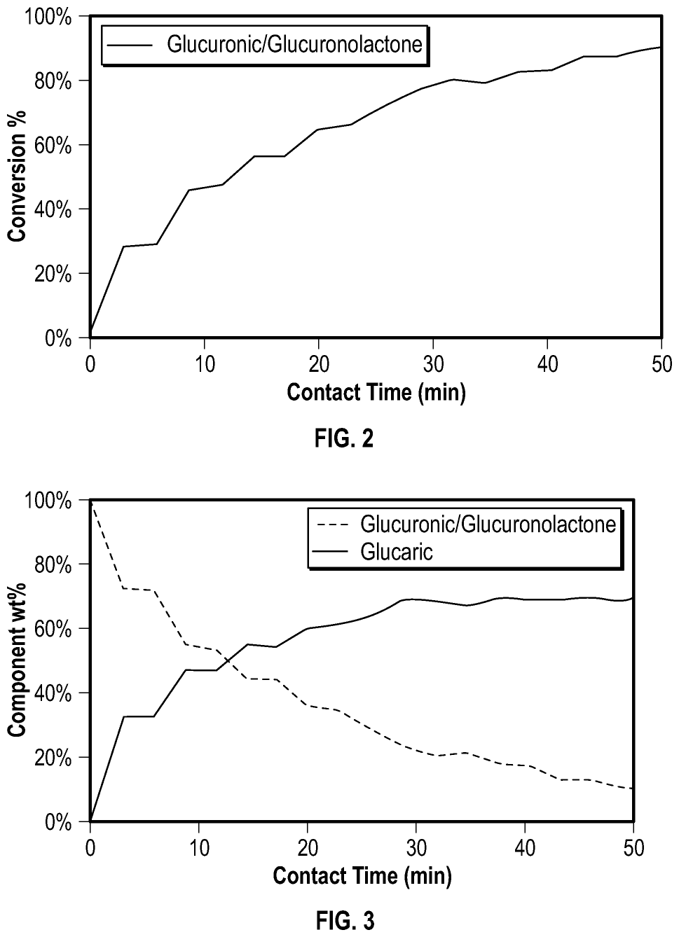

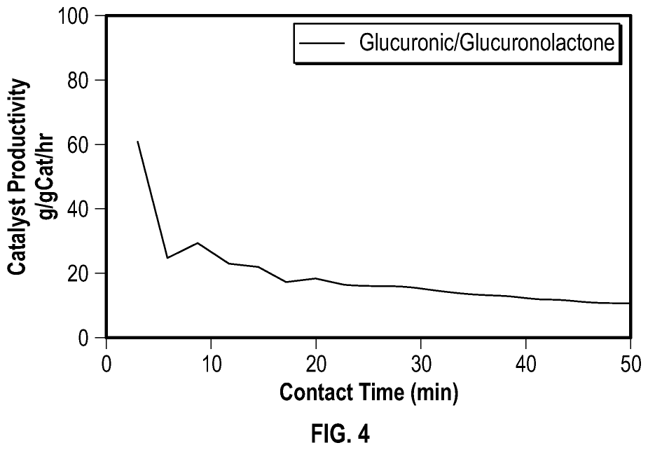

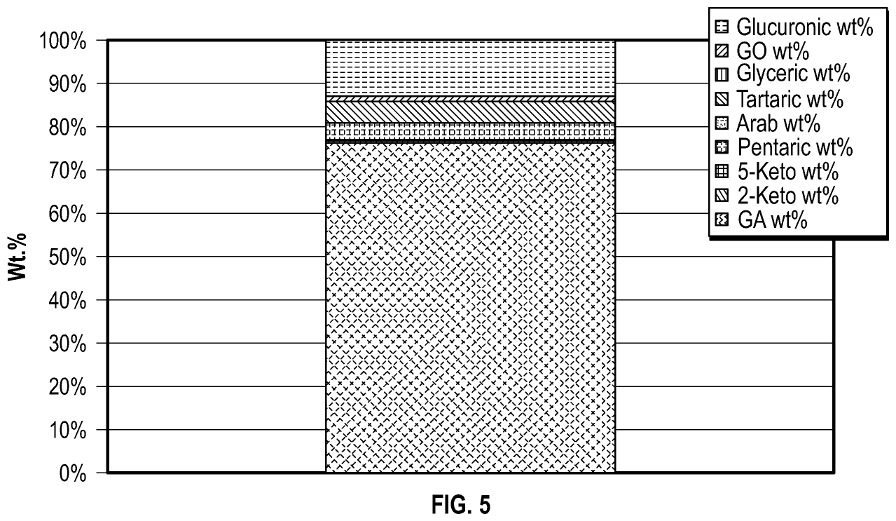

The HIGAP method of hydroxycarboxylic acid production was investigated using a pilot reactor experiment. Specifically, a proprietary platinum and gold-containing metal on carbon catalyst was loaded into a single pilot reactor vessel. A 15% glucuronolactone by weight in water feedstock was prepared as the primary feedstock for this experiment. Given the reactor size and catalyst loading amount, the experimental run was configured as a semi batch process with recycle passes conducted on the batch material. This multi-pass run was intended to mimic a large scale production with multiple reactors in series. The liquid feedstock was co-fed with air and heated to a temperature in the range of from about 110° C. to about-140° C. for a total of 8 reactor passes. In the tested reactor conditions glucuronolactone hydrolyzes to glucuronic acid. FIG. 2 shows the total conversion of the glucuronic/glucuronolactone feed on a mass basis. Wet mass percent measurements were collected via high performance liquid chromatography-mass spectrometry (HPLC-MS) quantitative methods. The HPLC-MS measurements were used to calculate a variety of other performance and composition metrics, including those in FIGS. 3 and 4. FIG. 3 tracked the dry weight percent content of glucuronic/glucuronolactone and glucaric in the process stream. FIG. 4 tracks the cumulative productivity of the reactor catalyst based on run time and catalyst exposure while FIG. 5 depicts the product distribution on a weight percent basis.

### Additional Disclosure

The following enumerated aspects of the present disclosures are provided as nonlimiting examples.

A first aspect which is a system for glucaric acid production, the system comprising: (a) a first input selected from the group consisting of glucuronolactone, a disaccharide feedstock, a cleaved starch, a disaccharide, glucuronic acid, or a combination thereof; (b) a first catalyst system comprising a metal oxidation catalyst; (c) a first product; (d) a second input; (e) a second catalyst system comprising an enzyme; and (f) a second product comprising from about 50% to about 99% glucaric acid on a dry basis.

A second aspect which is the system of the first aspect wherein the second product comprises equal to or greater than about 75% glucaric acid on a dry basis.

A third aspect which is the system of any of the first through second aspects wherein the first metal oxidation catalyst comprises a transition metal.

A fourth aspect which is the system of any of the first through third aspects wherein the first metal oxidation catalyst comprises one or more noble metals.

A fifth aspect which is the system of any of the first through fourth aspects wherein the first metal oxidation catalyst comprises gold.

A sixth aspect which is the system of any of the first through fifth aspects wherein the first input comprises a disaccharide selected from the group consisting of sucrose, lactose, maltose, isomaltose, isomaltulose, trehalose, trehalulose, and a combination thereof.

A seventh aspect which is the system of any of the first through fifth aspects wherein the first input comprises a disaccharide and the first output comprises a carboxylic disaccharide, a dicarboxylic disaccharide, or a combination thereof.

An eighth aspect which is the system of any of the first through fifth aspects wherein the first input comprises glucuronic acid, glucuronolactone, or a combination thereof.

A ninth aspect which is the system of any of the first through eighth aspects wherein the first product comprises an oxidized form of the first input.

A tenth aspect which is the system of any of the first through ninth aspects wherein the first product and the second input are the same.

An eleventh aspect which is the system of any of the first through tenth aspects wherein the second catalyst system comprises α-amylase, glucoamylase, β-glucuronidase, β-glucosidase and invertase, glucose oxidase, catalase, xylose isomerase, or a combination thereof.

A twelfth aspect which is a method for glucaric acid production, the method comprising (a) providing a first input selected from the group consisting of glucuronolactone, a disaccharide feedstock, a cleaved starch, a disaccharide, glucuronic acid, and a combination thereof; (b) contacting the first input with a first catalyst system comprising a noble metal oxidation catalyst; (c) producing a first product based on the contacting in step (b); (d) contacting a second input with a second catalyst system comprising an enzyme; and (e) producing a second product comprising from about 50% to about 99% glucaric acid on a dry basis based on the contacting in step (d).

A thirteenth aspect which is the method of the twelfth aspect wherein the second product comprises equal to or greater than about 75% glucaric acid on a dry basis.

A fourteenth aspect which is the method of any of the twelfth through thirteenth aspects wherein the first metal oxidation catalyst comprises gold, platinum, or a combination thereof.

A fifteenth aspect which is the method of any of the twelfth through fourteenth aspects wherein the first input comprises a disaccharide selected from the group consisting of sucrose, lactose, maltose, isomaltose, isomaltulose, trehalose, trehalulose, and a combination thereof.

A sixteenth aspect which is the method of any of the twelfth through fifteenth aspects wherein the first input comprises a disaccharide and the first output comprise a carboxylic disaccharide, a dicarboxylic disaccharide, or a combination thereof.

A seventeenth aspect which is the method of any of the twelfth through sixteenth aspects wherein the first input comprises glucuronic acid, glucuronolactone, or a combination thereof.

An eighteenth aspect which is the method of any of the twelfth through seventeenth aspects wherein the first product comprises an oxidized form of the first input.

A nineteenth aspect which is the method of any of the twelfth through eighteenth aspects wherein the first product and second input are the same.

A twentieth aspect which is the method of any of the twelfth through nineteenth aspects wherein the second catalyst system comprises α-amylase, glucoamylase, β-glucuronidase, β-glucosidase and invertase, glucose oxidase, catalase, xylose isomerase, or a combination thereof.

The subject matter having been shown and described, modifications thereof can be made by one skilled in the art without departing from the spirit and teachings of the subject matter. The aspects described herein are exemplary only and are not intended to be limiting. Many variations and modifications of the subject matter disclosed herein are possible and are within the scope of the disclosed subject matter. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.). Use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, etc. In addition, the phrases “a combination thereof,” “combinations thereof,” and “any combination thereof” following a list of recited items means any combination of two or more of the recited items in the list.

Accordingly, the scope of protection is not limited by the description set out above but is only limited by the claims which follow, that scope including all equivalents of the subject matter of the claims. Each and every claim is incorporated into the specification as an aspect of the present disclosure. Thus, the claims are a further description and are an addition to the aspects of the present invention. The discussion of a reference herein is not an admission that it is prior art to the presently disclosed subject matter, especially any reference that may have a publication date after the priority date of this application. The disclosures of all patents, patent applications, and publications cited herein are hereby incorporated by reference, to the extent that they provide exemplary, procedural, or other details supplementary to those set forth herein.

1. A system for glucaric acid production, the system comprising:
(a) a first input selected from the group consisting of glucuronolactone, a disaccharide feedstock, a cleaved starch, a disaccharide, glucuronic acid, or a combination thereof;
(b) a first catalyst system comprising a metal oxidation catalyst;
(c) a first product;
(d) a second input;
(e) a second catalyst system comprising an enzyme; and
(f) a second product comprising from about 50% to about 99% glucaric acid on a dry basis.

2. The system of claim 1, wherein the second product comprises equal to or greater than about 75% glucaric acid on a dry basis.

3. The system of claim 1, wherein the first metal oxidation catalyst comprises a transition metal.

4. The system of claim 1, wherein the first metal oxidation catalyst comprises one or more noble metals.

5. The system of claim 1, wherein the first metal oxidation catalyst comprises gold.

6. The system of claim 1, wherein the first input comprises a disaccharide selected from the group consisting of sucrose, lactose, maltose, isomaltose, isomaltulose, trehalose, trehalulose, and a combination thereof.

7. The system of claim 1, wherein the first input comprises a disaccharide and the first output comprises a carboxylic disaccharide, a dicarboxylic disaccharide, or a combination thereof.

8. The system of claim 1, wherein the first input comprises glucuronic acid, glucuronolactone, or a combination thereof.

9. The system of claim 1, wherein the first product comprises an oxidized form of the first input.

10. The system of claim 1, wherein the first product and the second input are the same.

11. The system of claim 1, wherein the second catalyst system comprises α-amylase, glucoamylase, β-glucuronidase, β-glucosidase and invertase, glucose oxidase, catalase, xylose isomerase, or a combination thereof.

12. A method for glucaric acid production, the method comprising:
(a) providing a first input selected from the group consisting of glucuronolactone, a disaccharide feedstock, a cleaved starch, a disaccharide, glucuronic acid, and a combination thereof;
(b) contacting the first input with a first catalyst system comprising a noble metal oxidation catalyst;
(c) producing a first product based on the contacting in step (b);
(d) contacting a second input with a second catalyst system comprising an enzyme; and
(e) producing a second product comprising from about 50% to about 99% glucaric acid on a dry basis based on the contacting in step (d).

13. The method of claim 12, wherein the second product comprises equal to or greater than about 75% glucaric acid on a dry basis.

14. The method of claim 12, wherein the first metal oxidation catalyst comprises gold, platinum, or a combination thereof.

15. The method of claim 12, wherein the first input comprises a disaccharide selected from the group consisting of sucrose, lactose, maltose, isomaltose, isomaltulose, trehalose, trehalulose, and a combination thereof.

16. The method of claim 12, wherein the first input comprises a disaccharide and the first output comprise a carboxylic disaccharide, a dicarboxylic disaccharide, or a combination thereof.

17. The method of claim 12, wherein the first input comprises glucuronic acid, glucuronolactone, or a combination thereof.

18. The method of claim 12, wherein the first product comprises an oxidized form of the first input.

19. The method of claim 12, wherein the first product and second input are the same.

20. The method of claim 12, wherein the second catalyst system comprises α-amylase, glucoamylase, β-glucuronidase, β-glucosidase and invertase, glucose oxidase, catalase, xylose isomerase, or a combination thereof.
